(19) United States
(12) Patent Application Publication
(10) Pub. No.: US 2019/0203250 A1
(43) Pub. Date: Jul. 4, 2019

(54) MICROBIAL PRETREATMENT FOR CONVERSION OF BIOMASS INTO BIOGAS

(71) Applicants: JARON C. HANSEN, SPRINGVILLE, UT (US); LEE D. HANSEN, SARATOGA SPRINGS, UT (US); ZACHARY T. AANDERUD, PROVO, UT (US); CONLY L. HANSEN, N. LOGAN, UT (US)

(72) Inventors: JARON C. HANSEN, SPRINGVILLE, UT (US); LEE D. HANSEN, SARATOGA SPRINGS, UT (US); ZACHARY T. AANDERUD, PROVO, UT (US); CONLY L. HANSEN, N. LOGAN, UT (US)

(21) Appl. No.: 16/194,271

(22) Filed: Nov. 16, 2018

Related U.S. Application Data
(60) Provisional application No. 62/587,417, filed on Nov. 16, 2017.
(60) Provisional application No. 62/750,221, filed on Oct. 24, 2018.

(51) Int. Cl. C12P 39/00; C12M 1/00; C12M 1/33; C12M 1/107

(52) U.S. Cl. CPC C12P 39/00; C12M 45/06; C12M 21/04; C12M 45/09; C12M 45/02

(57) ABSTRACT

A system for degrading biomass with anaerobic digestion that includes a biological pretreatment with organisms that break down lignocellulosic materials before anaerobic digestion or for use as feedstock for other reactions.

### BACKGROUND

Lignocellulosic biomass is a relatively inexpensive, renewable and abundant material that can be used to generate fuels, chemicals, fibers, and energy. Large-scale production of lignocellulosic products is hindered, at least in part, by the lack of low-cost technologies capable of efficiently converting the lignocellulosic biomass into soluble, reactive intermediates.

Anaerobic digestion is a process that has been explored to readily and rapidly convert soluble products into biogas with 60-70% methane. Isothermal biomicrocalorimetry measurements have provided a thermodynamic understanding of the process. Pretreatment of lignocellulosic biomass prior to anaerobic digestion renders the biomass more amenable to anaerobic digestion. To that end, purely mechanical and purely chemical means have both been proposed as ways to perform pretreatment. But even where improvements of the anaerobic digestion have been shown, there have been significant drawbacks. For example, purely mechanical means are energy intensive, uneconomical, and only modestly effective. Purely chemical means introduce agents such as acids or bases that are generally poisonous to the anaerobic bacteria, and any advantages from breaking down the biomass components by the chemicals are compromised by creating a less than optimal environment in the anaerobic digestion tank. Chemical means further create a disposal problem for toxic wastes that result.

Without pretreatment of lignocellulosic biomass, anaerobic digestion of lignocellulosic biomass typically converts only one-third of the carbon into biogas which is typically only 60% methane. While anaerobic digestion by microorganisms is effective on hemicellulose-side chains, cellulose, long glucose chains, are only slowly digested by anaerobic digestion microorganisms, and lignin, a polyphenol, is resistant or toxic to many microorganisms. Improvements to pretreatment of biomass are thus gaining further attention.

### BRIEF DESCRIPTION OF DRAWINGS

FIG. 1 is a diagram of an example reactor according to principles described herein.

FIG. 2 is a diagram of an example reactor according to principles described herein.

FIG. 3 is a diagram of an example reactor according to principles described herein.

FIG. 4 is a diagram of an example reactor according to principles described herein.

FIG. 5 is a diagram of an example reactor according to principles described herein.

FIG. 6 is a diagram of an example reactor according to principles described herein.

FIG. 7 is a diagram of an example reactor according to principles described herein.

FIG. 8 is a diagram of an example reactor according to principles described herein.

FIG. 9 is a diagram of an example reactor according to principles described herein.

FIG. 10 is a diagram of an example reactor according to principles described herein.

FIG. 11 is a diagram of an example reactor according to principles described herein.

### DETAILED DESCRIPTION

The following describes the introduction of bacteria to reactors and various components of reactors that may be used in the course of biological pretreatment and anaerobic digestion of biomass.

Pretreating biomass prior to anaerobic digestion with a bacteria or organism capable of breaking down lignocellulose (e.g., cellulose, hemicellulose, lignin, bacteria, etc.) has been shown to be effective in promoting anaerobic digestion and does not introduce any harmful chemicals or otherwise harm the environment for the anaerobic bacteria in the anaerobic digestion yielding biogas.

A recent study indicates that the anaerobic thermophile Caldicellulosiruptor bescii (“C. bescii”) solubilizes biomass. C. bescii has been shown to be capable of solubilizing up to 90% of lignocellulose and cellular material, thus making the carbon accessible for anaerobic digestion.¹ Such a reaction supports the notion of industrial conversion of biomass by extremely thermophilic microbes by pretreatment that requires limited or no chemical additions and that may be useful as a pretreatment. Preliminary experiments by researchers at Brigham Young University show that C. bescii is capable of solubilizing a wide range of lignocellulosic materials and materials composed of cellular material. ¹ “Carbohydrate and lignin are simultaneously solubilized from unpretreated switchgrass by microbial action at high temperature,” Energy and Environmental Science, Issue 7, 2013.

Pretreatment-anaerobic digestion has been applied to giant king grass, mixed green waste, paper, and several other feedstocks on a pilot plant scale, and it is expected that prototypes for pretreatment processing with C. bescii will scale to commercial anaerobic digestion systems, such as electrical generation on a megawatt scale.

The term “tank” refers to an actual tank or other reaction containment, space or environment in which suitable conditions are met. The term further includes a suitable continuous or semi-continuous flow reactor, plug-flow reactor, reaction tank, etc.

The term “reactor” refers to at least one or more of tanks and components of tanks used in relation to the pretreatment and treatment of lignocellulosic biomass.

The term “environment” is used to anticipate environments that include not only tanks and reservoirs, but other environments for reactor principles discussed herein.

The term “mixing tank” refers to a tank that heats and mixes biomass with one or more of water and reagents.

The term “anaerobic secretome bioreactor” (“ASB tank”) refers to a tank that is used for pretreatment of biomass in anaerobic conditions and that produces ASB effluent comprising at least one or more of a solubilized biomass, liquid effluent, and solid effluent.

The term “anaerobic digestion tank” (“AD tank”) refers to a tank that is used to receive the ASB effluent and break it down under anaerobic conditions with at least one of the products being biogas which may be combusted to generate electricity and heat, or further processed into renewable natural gas and transportation fuels.

The term “satellite reservoir” refers to a reservoir that is configured to maintain and provide a microbe, nutrient solution, or pH adjusting chemicals to at least one or more of an ASB tank and an AD tank.

### Mixing Tank

The mixing tank comprises a tank that mixes biomass with water, and possibly other reagents as well. The contents may then be heated within the tank. Alternatively, one or more of the contents may be heated prior to entry within the mixing tank. Contents introduced having lignocellulosic biomass may include one or more of animal waste, human waste, food waste/garbage, organic matter, plant matter (e.g., green waste, bio-energy crops, algae, coconut husk, grass, etc.), waste activated sludge, and algae grown in reactors. Lignocellulosic biomass along with other types of raw material or feedstock may be introduced into the mixing tank being pre-mixed together or added separately.

Besides lignocellulosic biomass, alternatives include biomass and waste paper that does not have lignin, such as slaughterhouse waste and waste paper. Alternatives further include both lignocellulosic and non-lignocellulosic biomass.

An example treatment includes the biomass being ground to a 3 cm particle size and then being supplied to the mixing tank before being introduced to the ASB tank. The contents of the mixing tank are adjusted to a composition that is approximately 2% to 50% (e.g, 6% has been found suitable), and heated to approximately 60 ° C. to 100 degrees ° C. for 1 to 6 hours. During this period of time, mixing can be accomplished, for example, with one or more of a plurality of paddles and/or pumps. This process has the effect of pasteurizing the contents, expelling dissolved O₂, and providing the contents with an optimal temperature for thermophilic microbial action.

The resultant mixing tank effluent is sent to the ASB tank in the form of a liquid suspension, slurry, or mash of solids. An example solids content of the effluent may represent approximately 10% of the effluent.

Alternatively or in addition, a solution or “tea” containing soluble materials in the biomass may be separated from the solids content and be sent directly to an AD tank.

For some types of biomass, raw material, and feedstocks, the mixing tank also serves as a hydration tank in which hydration is provided. Also, two separate tanks are anticipated, one tank for hydration and one tank for mixing.

### ASB Tank

The mixing tank effluent from the mixing tank is directed into the ASB tank, for example, at levels approximating 10% of the influent solids content. Within the ASB tank, the mixing tank effluent receives exposure to at least one material comprising a thermophilic anaerobic microbe, such as C. bescii, a bacteria that is provided in the ASB tank. The bacteria “solubilizes” at least a portion of the biomass effluent, essentially breaking down plant cell walls within the material and making contents of the plant cells available for subsequent anaerobic digestion.

Contents introduced having lignocellulosic biomass may include one or more of animal waste, human waste, food waste/garbage, organic matter, plant matter (e.g., green waste, bio-energy crops, algae, coconut husk, grass, etc.), waste activated sludge, and algae grown in reactors. Lignocellulosic biomass along with other types of raw material or feedstock may be introduced into the ASB tank being pre-mixed together or added separately.

After pretreatment, the ASB effluent leaves to the AD tank or other location. The ASB effluent from the ASB tank may further be concentrated and purified to serve as a feedstock or reagent for other processes. To meet the conditions of the AD tank, the ASB effluent may be cooled, for example, by a cooling reservoir or heat exchange system, prior to entering an AD tank or other location.

During the pretreatment process within the ASB tank, the ASB tank is maintained at a desired temperature to provide a suitable environment for the C. bescii or other microbes to solubilize cellulose. An example temperature may be kept at 75° C. or approximately 75° C. For example, a temperature range may be maintained between 55° C. to 85° C. Narrower ranges include 55-60° C., 60-65° C., 65-70° C., 70-75° C., 75-80° C., 80-85° C., 60-70° C., 70-80° C., 70-85° C., or 60-85° C. degrees, or other ranges that are used for pasteurization and growth of an anaerobic microbe.

The ASB tank is heated to maintain the temperature using any suitable means. Heat may be suitably recycled from other processes such as waste heat from engines and other mechanical devices, exhaust, combustion gas heat, solar, etc.

Another condition to consider for the ASB tank is the oxygen limit. The ASB tank is configured to adhere to oxygen limits for C. bescii, based on C. bescii being a strict anaerobe, which is pO₂=0.5%. The ASB may further be configured for higher oxygen levels. For example, C. bescii is capable to withstand oxygen levels as high as pO₂=20% for 15-20 minutes.

Another condition for the ASB tank is a suitable pH that should be maintained for the ASB bacterial growth, such as a range between 4.5 to 8.5. For optimal growth of C. bescii, the pH should be controlled between 6.5 and 8.5, or 6.8 and 7.2. Lower pH values do not immediately kill bacteria, but slowly starve it because it cannot acquire energy by metabolizing sugars from dissolved cellulose. Dissolution of the cellulose also stops because the products are not being metabolized.

Another condition for the ASB tank is having sufficient base. Sufficient base must always be present to react with the acids produced in metabolism. Metabolism and growth of C. bescii are driven by production of CO₂ gas or water. A base that does not produce sufficient Gibbs energy upon reaction with acids will not provide the energy required for optimum growth of the bacteria. A base may be introduced for pH control and promotion of metabolism. This can be accomplished by introducing a bicarbonate (e.g., HCO₃⁻, etc.), or recycling bicarbonate from the AD as further described below.

C. bescii is believed to not form a biofilm on biomass particles. Instead, it produces exozymes that catalyze dissolution of the lignocellulosic materials. The ASB tank may be stirred for C. bescii to increase contact between bacteria and biomass and support the production of exozymes that dissolve the lignocellulosic materials.

An exemplary mixing system may comprise a slow or low-level mechanical stirring or other suitable system for mixing. Means for mixing the contents may include one or more of a plurality of paddles and pumps.

Pretreatment may be one or more batch process, semi-continuous process, and a continuous process. The reaction in the ASB tank may depend on the time period depending on the feedstock and desired level of material destruction. In an example, ASB containment for reaction includes a period of time between 0 to 48 hours. In another example, the period of time is 0 to 7 days.

The time for a reaction using C. bescii may be between 0.5 hour to 200 hours, but more typically 12-72 hours. C. bescii is suitable because it can rapidly depolymerize and solubilize lignocellulose (plant material) and other cellular material. C. bescii further produces exozymes that catalyze hydrolysis of cellulose and lignin at a rapid rate. The products are sugars and phenolic compounds from lignin. Sugars are metabolized to acetic acid and lactic acid by C. bescii as a source of Gibbs energy for growth and activity. Phenolics are believed not to be metabolized by C. bescii. Some of the phenolic compounds that are formed include 4-hydroxybenzoic acid, caffeic acid, p-coumaric acid, ferulic acid, phenol, and benzoic acid.

Pretreatment Reactions with C. bescii Include:

Lignocellulose→(acetate+lactate) ions+CO₂(g)+oxygenated aromatics from lignin+residual sugars. These reactions are catalyzed by exozymes produced by C. bescii. During pretreatment in the ASB tank, acetic and lactic acids in the presence of bicarbonate react to produce acetate ion, lactate ion, and CO₂ gas. Acetic and lactic acids react with base in the pH buffer to produce acetate ion, lactate ion, and CO₂ gas. The rate of pretreatment can be obtained by monitoring the increase in CO₂ gas pressure over the pretreatment mixture in a sealed vessel or by one or more of measurement of the change in total suspended solids and volatile solids as pretreatment progresses. Products are not toxic to anaerobic digestion microorganisms and are rapidly digested in the AD tank to produce biogas.

Pretreatment metabolic reactions include:

Sugars→acetic acid+lactic acid. For this reaction to occur at a significant rate, the concentration of acetic and lactic acid must be kept low by reaction with bicarbonate or another base.
Acetic acid+bicarbonate→acetate ion+CO₂+H₂O. The Acetate/Lactate ions and residual sugars are rapidly metabolized in the AD by anaerobic bacteria, producing CO_{2,} and CH₄, as follows;
Acetate/Lactate ions+sugars→methane+CO₂+bicarbonate (which may be recycled back to the ASB tank).

One or more bacterial organisms used in the ASB tank can be introduced by any suitable methods, and conditions should be maintained for optimum growth. For example, trace elements, nutrients, vitamins, and the like may be introduced to start or maintain the pretreatment process.

For C. bescii, specific optimum conditions have been shown to include the following: 78° C., DSMZ media (0.33 g NH₄Cl, 0.33 g KH₂PO₄, 0.33 g KCl, 0.33 g MgCl₂*6 H₂O, 0.33 g CaCl₂*2 H₂O, 0.50 g Yeast Extract, 0.50 mL Na-resazurin solution (0.1% w/v), 1.50 g NaHCO₃, 0.50 g Na₂S*9 H₂O, 1.00 mL of a trace element solution, 10.00 mL of a vitamin solution to 1000.00 mL distilled water.

The trace element solution (designated SL-10) was, in an example, composed of 10.00 mL HCl (25%; 7.7 M), 1.50 g FeCl₂*4 H₂O, 70.00 mg ZnCL2, 100.00 mg MnCl₂*4 H₂O, 6.00 mg H₃BO₃, 190.00 mg CoCl₂*6 H₂O, 2.00 mg CuCl₂*2 H₂O, 24.00 mg NiCl₂*6 H₂O, 36.00 mg Na₂MoO₄*2 H₂O, and 990.00 mL Distilled water. The FeCl₂ was dissolved in the HCl, which was then diluted in water. The remaining salts were added and the solution was diluted to 1000.0 mL. The vitamin solution was composed of 2.00 mg Biotin, 2.00 mg Folic acid, 10.00 mg Pyridoxine-HCl, 5.00 mg Thiamine-HCL*2 H20, 5.00 mg Riboflavin, 5.00 mg Nicotinic acid, 5.00 mg D-Ca-pantothenate, 0.10 mg Vitamin B12, 5.00 mg Lipoic Acid, and 1000.00 mL Distilled water. The solutions should be stored under anaerobic conditions.

In addition to C. bescii, other organisms may be suitable for the ASB tank. These include, but are not limited to other bacteria, fungi, archea, genetically modified bacteria, cellular organisms, and any organism or mixture of organisms with suitable properties for digesting lignocellulosic materials. These organisms may be found in hot springs and/or concentrations of rotting wood, include lignocellulose-degrading extremophiles. Possible candidates for the ASB tank include other bacteria of one or more of the genus Caldicellulosiruptor, Clostridium thermocellum, and Thermoanaerobacterium saccharolyticum, and other bacteria.

A great expense may be involved in hauling away and safely disposing of waste material. The ASB tank and other components described herein have great market potential because of the increase in quantity of commercially viable product that they yield over current methods. Another advantage in using the ASB tank and other components is that they yield 1.5 to 10 times as much gas than if biomass is put directly into an AD tank.

### AD Tank

The AD tank receives the ASB effluent from the ASB tank and operates essentially as a typical anaerobic digester found in current practice, except the AD tank is fed by the ASB tank. Compared to conventional feed streams of an anaerobic digester, the ASB effluent is more available for anaerobic metabolism and digestion than typical biomaterials.

The AD tank has two main product streams which include gas and liquid forms. The first main output stream, or biogas stream, contains mostly CH4 and CO2, but also other reaction products and impurities (e.g., H2S, and H₂O). The biogas stream is directed to suitable gas processing for its intended use. The second main output stream is a slurry of undigested biomass, which can be processed as a soil conditioner or compost.

Within the AD tank are maintained suitable bacteria, such as acetogens and methanogens, that support production of biogas. Acetogens and methanogens can grow better with the ASB effluent from the ASB tank than with conventional untreated biomass.

Conditions in the AD tank are maintained to allow anaerobic bacteria to thrive. An example temperature includes 40 degrees C., however the temperature may range between 15° C. to 85° C. The pH of the AD tank may range from 6.5 to 8.5, with more narrow ranges including 6.5-7, 7-.7.5, 7.5-8, or 8-8.5. The temperature and pH are conditions that are far from the conditions in the ASB tank. Maintaining disparate conditions where an organism of the ASB tank and the bacteria in the AD tank can thrive is a primary reason that separate tanks or digesters are provided for each part of the process.

The AD tank may include a continuous stir, up flow anaerobic sludge blanket, induced bed reactor, a dual anaerobic/aerobic digestor, or any kind of anaerobic digestor for the reaction. Treatment may be one or more batch process, semi-continuous process, and a continuous process.

A heat exchanger may be used to harvest excess heat from the effluent that leaves the ASB tank and recycle the heat back to the ASB tank, mixer tank, or other location. Recycling heat back to the ASB tank or mixer tank is advantageous in helping maintain their respective temperatures. For an ASB tank that is typically about 75° C. and an AD tank that is typically about 40° C., the heat that can be directed to the ASB tank is significant.

Reactions in the AD tank include:

(Acetate) CH₃COO⁻_{(aq)}→CH_{4(g)}+HCO₃⁻_{(aq)}

(Lactate) 2CH₃CH(OH)COO⁻_{(aq)}+H₂O→3CH_{4(g)}+2HCO₃⁻_{(aq)}+CO_{2(g)}

(Sugars) 2C₁H₂O→CH_{4(g)}+CO_{2(g)},

Note that production of acetate in the ASB tank uses one bicarbonate and digestion of acetate produces one bicarbonate in the AD tank. Production of lactate uses one bicarbonate and digestion of lactate produces one bicarbonate ion. Thus, the reactions in the AD tank produce the same amount of bicarbonate as the amount used in pretreatment in the ASB tank. Balancing bicarbonate (i.e. acid/base balance) would require recycling 100% of the biomass effluent from the AD tank. Efficiency of that nature is unlikely to be possible and not practical. Instead, bicarbonate may be introduced to the ASB tank or the mixing tank from other sources, or by adding another base to the ASB tank, such as ammonia, sodium carbonate, sodium bicarbonate, potassium hydroxide, and sodium hydroxide.

### Biogas Conditioner

If required, gas taken from the AD tank may be subjected to a biogas conditioner, which is a method designed to process or condition gas suitable for its intended use. This method may involve removal of CO₂, which can be optionally recycled to one or more of the mixing tank, ASB tank, and AD tank to displace oxygen or air.

Gas processing requirements may be significantly reduced or eliminated by choice of the ASB bacteria and/or the processing conditions. As noted above, lactate in the AD tank will be metabolized to methane (CH₄), bicarbonate (HCO₃⁻), and carbon dioxide (CO₂). Acetate is metabolized to methane and bicarbonate. Sugars will be metabolized to methane and carbon dioxide. If carbon dioxide is reduced or eliminated as an AD tank product, gas processing to remove carbon dioxide can be correspondingly reduced or eliminated.

An experiment was conducted to test the modification of the bacteria and/or conditions in the ASB tank for production of only acetate instead of a mixture of acetate, lactate, and sugars. With only acetate as the feed to the AD tank, modification of the AD bacteria and/or conditions would enable the production of methane with little or no CO2 in the biogas. This would eliminate gas processing to remove CO2, which is a costly process. In the experiment, it was shown that it was possible to produce only acetate by pretreatment with C. bescii. When that solution was passed on to the AD tank, the biogas was 85% methane and 15% CO2. The source of the CO2 was not determined, but it probably came from compounds other than acetate that were produced in the ASB tank, but not measured when the acetate was measured. Lactate and glucose were found to be absent from the solubilized biomass from the ASB tank.

A suitable use for the biogas produced is power generation. This would involve combustion of the CH4. The combustion gas, which contains mostly nitrogen, but also CO2, may also advantageously be recycled to the Mixing Tank or the ASB tank to displace oxygen or air.

Another suitable use for the biogas is to have the biogas processed and compressed for CNG. It may also be used as a feed stock for chemical processing, such as a Fischer-Tropsch process for production of biodiesel or other fuel.

To increase process efficiency, reduce material costs, and reduce parasitic losses of material and energy, suitable recycling streams and heat recovery systems can be employed. These include recovering heat using, for example, heat exchangers from the ASB effluent from the ASB tank, from the mixing tank effluent from the mixing tank, combustion gasses, or other hot downstream products. The recovered heat from any of these sources may be combined or used separately. They may be used to heat the mixing tank, the ASB tank, or heat process streams going into these systems. In one example, heat can be recovered from the ASB effluent leaving the ASB tank and the mixing tank effluent from the mixing tank and used to preheat water that is used for the mixing tank. Bicarbonate from the AD tank or other base may be recycled to maintain growth conditions in the ASB tank.

Further recycling may be to remove oxygen. For example, the AD and ASB tanks are anaerobic and oxygen can be removed from the AD and ASB tanks and any input streams, for example, by flushing the AD and ASB tanks with CO2 from combustion or gas processing.

In addition to recycling, exemplary pretreatment processes may further include purification treatments. For example, contents received within the ASB tank may first be processed through a purification processing treatment. Recycled matter from the AD tank may go through a purification processing treatment before being received by the ASB tank. Materials from the AD tank may be purified before being separated into different elements or before being used to produce biogas.

### ASB Satellite Reservoir

There may be times where the ASB tank becomes compromised due to a reinoculation failure, chemical restriction of the feedstock presented, or other problem. To counter occurrences of this nature, an ASB satellite reactor may be used to maintain and provide one or more of bacteria, nutrient, or other matter to the ASB tank.

For example, an exemplary satellite facilitates the continuous inoculum of C. bescii. In addition, to containing one or more of active C. bescii, the satellite may contain nutrients necessary for C. bescii growth, and a small amount of the pretreatment feedstock (e.g., biosolids, green waste, energy crops, food waste, raw or organic materials, etc.). The C. bescii or other matter in the reactor should be maintained at or near 1×10⁶ cell density.

Addition of the satellite reservoir may fulfill at least five primary needs: 1) it maintains ASB bacteria culture, 2) it alleviates the need for trace elements to be added to the main ASB tank depending on feedstock chemical characteristics, 3) it conditions C. bescii to utilize the feedstock, 4) it efficiently acts as a back-up to reinoculate a failed or compromised ASB tank, and 5) it speeds up the pretreatment process in the ASB tank by avoiding time that otherwise would be required for the C. bescii to grow in the ASB tank. The satellite reservoir may also be used to add a base such as bicarbonate to the ASB tank to maintain pH and support metabolism.

The ASB satellite reservoir has a volume ratio to the ASB tank within a range of 1:10 to 10:1. Alternatively, the volume of the ASB satellite reservoir is approximately 1/100 or within a range of 1/200 to 1/2 of the volume of the ASB tank. Also, the satellite reservoir may contain 0.5-1.0% of the pretreatment feedstock. An example satellite reservoir requires that the entire volume of the reservoir be replaced every 72 hours or a within range of 50-80 hours. Example nutrients maintained in the satellite reservoir include, per liter of satellite reservoir: glucose (1 g), yeast extract (0.1 g) (e.g., brewer's yeast or another source of amino acids, etc.), NH₄Cl (0.05 g), KH₂PO₄ (0.05 g), MgCl (0.05 g), CaCl2 (0.05 g), NaHCO₃ (1.0 g), and Na₂S (0.1 g to ensure anaerobic conditions).

Maintaining a specific temperature or temperature range keeps the tank inoculated with a particular bacteria. Temperature ranges that are maintained in the satellite reservoir may include one or more of 60-65° C., 65-70° C., 70-75° C., 75-80° C., 80-85° C., 60-70° C., 70-80° C., 70-85° C., or 60-85° C. degrees. Temperature changes should be minimized during material transport from the satellite reservoir to the ASB tank.

Example trace elements that may be provided are shown in the following table:

| Trace element solution SL-10: | | |
|---|---|---|
| HCl (25%; 7.7M) | 10.00 | ml |
| FeCl₂ × 4 H₂O | 1.50 | g |
| ZnCl2 | 70.00 | mg |
| MnCl₂ × 4 H₂O | 100.00 | mg |
| H₃BO₃ | 6.00 | mg |
| CoCl₂ × 6 H₂O | 190.00 | mg |
| CuCl₂ × 2 H₂O | 2.00 | mg |
| NiCl₂ × 6 H₂O | 24.00 | mg |
| Na₂MoO₄ × 2 H₂O | 36.00 | mg |
| Distilled water | 990.00 | ml |

First dissolve FeCl₂ in the HCl, then dilute in water, add and dissolve the other salts. Finally make up to 1000.0 ml.

Trace nutrients or trace elements are maintained in the satellite reservoir that are necessary for a bacteria in the ASB tank to grow and divide. An example operation includes the addition of trace nutrients and trace elements provided from the ASB satellite reservoir to the ASB tank to overcome the chemical restrictions of the feedstock. In operations that include C. bescii, one or more of additional nutrients and trace metals may need to be provided to further supply the ASB tank in order to facilitate C. bescii growth.

In addition to trace nutrients and trace elements, sucrose or a carbon source may be maintained in the satellite reactor. Bacteria besides C. bescii that may be maintained in the satellite reactor includes one or more of caldicellulosiruptor bescii , Caldicellulosiruptor genus, Clostridium thermocellum, Thermoanaerobacterium saccharolyticum. The bacteria in the reactor is maintained at a pH range of 6.5-7, 7-.7.5, 7.5-8, 8-8.5, 6.5-7.5, 7.5-8.5, or 6.5-8.5.

The satellite reservoir provides one or more of bacteria, nutrient, or other matter primarily at two different times. First, at the start of ASB tank pretreatment, contents from the ASB satellite reservoir are added if and when nutrients are needed for the feedstock to start to decompose and nutrients and trace elements are released from the substrate, usually occurring within 48-72 hours. A table of feedstock and the mean doubling time with C. bescii is shown below:

| Feedstock | C. bescii Mean Doubling Time (hours) |
|---|---|
| Starch | 2.1 |
| Newspaper | 4.6 |
| Barley Shoots | 3.6 |
| Kentucky Blue Grass | 3.4 |
| lawn clippings | |
| Switchgrass | 2.3 |
| Poplar twigs | 2.4 |
| Corn Husk | 4.3 |

An exemplary ASB tank is fed in such a way so as to maintain an exponential growth culture. The mean doubling times, such as the mean doubling time shown in the table, may be used to extract information on the way to feed the ASB tank so as to maintain an exponential growth culture.

An exemplary ASB tank is also fed so as to maintain growth of the organism within the ASB environment.

During the ASB tank pretreatment, if the feedstock lacks the necessary requirements for C. bescii growth, contents from the satellite reservoir provide specific chemical constituents. The C:N:P ratio of 500:10:1 dictates the addition of major nutrients, N and P, as NH₄lI, yeast extract, and/or KH₂PO₄. Note that an example satellite reservoir may provide one type of content or alternatively, may provide one or more select bacteria, nutrients, or other matter.

An example ASB satellite reservoir includes a monitoring system or device that monitors one or more of pH level, oxygen content, and type of bacteria present within the reactor. Also, the monitoring system or a second monitoring system may monitor both the ASB tank and/or AD tank. A delivery system manually, automatically, or semi-automatically delivers contents from the satellite reservoir to the ASB tank in quantities that are determined to be needed for the ASB tank. Chemical analysis may be performed so that trace metals that are lacking in the ASB tank may be added including one or more of Fe, Zn, Mn, B, Co Cu, and Ni. Product formation of acetate, lactate, oxygenated aromatic compounds, etc., is also monitored.

Additionally, the bacteria may be cultured in a separate small satellite reservoir to be inoculated.

The contents in an ASB satellite reservoir are grown on a substrate, contents, and/or conditions that are close or identical to the biomass that is pre-treated in the ASB tank. If the contents are only given one type of biomass, it will have a difficult time being able to break down other types of biomass. The reason for this is that bacteria do not make enzymes that are not needed. For example, growing bacteria with sucrose will shut down genes that are not needed to metabolize sucrose. Introducing bacteria fed with sucrose into an ASB tank with grass clippings will not enable the bacteria to break down the grass clippings because the bacteria were conditioned to metabolize sucrose. Maintaining similar conditions of bacteria and biomass between the ASB tank and the ASB satellite reservoir is therefore an advantage to the system as a whole.

Additionally, adding trace nutrients, sucrose, carbon source, etc. to the bacteria promotes optimal conditions for the bacteria to grow and thrive in the satellite reservoir and produce enzymes to be transported to the ASB tank. Alternatively, a yeast, such as brewer's yeast, may be added to promote growth of the bacteria by providing needed amino acids.

Oxygen levels within the ASB satellite reservoir are similar as the oxygen levels as the ASB tank. The ASB satellite reservoir is configured to adhere to the oxygen limits for C. bescii, based on C. bescii being a strict anaerobe, which is pO₂=0.5%. The satellite reactor may further be configured for higher oxygen levels, such as pO₂=2% for 15-20 minutes.

Although reference is made specifically to the ASB tank, the ASB satellite reservoir and its principles may be applicable to other tanks and processes described herein and are not intended to be limited to the ASB tank. For example, a satellite reservoir may be used to one or more of ASB tank, the AD tank, the mixing tank, and processes that occur during any phase related to reactions described herein.

### AD Satellite Reservoir

An advantage of extremely thermophilic pretreatment is pasteurization of the feedstock before it is introduced into the AD satellite reservoir, thus allowing better control of the AD microbes and process. An AD satellite reservoir may be connected to the AD tank to incubate bacteria that is used to augment the bacteria desired in the AD tank. For example, it may be useful to augment bacteria that are specific to biogas production from the molecules being produced in the ASB tank.

As an alternative, the AD tank may contain synthetic contents or biogenetically engineered contents which are bio-augmented. In this case, the ASB tank and the AD satellite reservoir would likewise contain respective synthetic contents or biogenetically engineered contents.

In an example, AD tanks are augmented from the AD satellite reservoir with an acetoclastic consortium isolated from WAS. Bioaugmentation with acetoclastic archaea has recently shown that within 7 days, bioaugmentation significantly reduced acetate accumulation and the proportion of methane in biogas increased almost over a hundred-fold (Town and Dumonceaux, 2015). The consortium may be isolated during multiple successful digestions of WAS when methane production is relatively high to increase the likelihood that the archaea will thrive within similar AD conditions. The consortium may be cultured with DSMZ Medium 141 to capture local methanogens and an additional consortium will be created that is spiked with two well-known Methanosarcina, Methanosarcina barkeri, DSMZ 800, and Methanosarcina acetivorans DSMZ 2834. Consortia may be cultivated continuously at 55° C. for 6 months prior to bioaugmentation experiments and produced with consistent levels of methane in biogas. Consortium may be added to bioreactors and vessels prior to WAS/AD and periodically to reseed the digesters when methane production drops.

In another example, the AD satellite reservoir supplies one or more of a nutrient solution and base to the AD tank.

An example of a reactor includes at least three satellite reservoirs, a first satellite reservoir that is configured with certain contents and a second satellite reservoir that is configured with certain different contents. For example, the first satellite reservoir includes C. bescii, the second satellite reservoir includes acetoclastic bacteria, and the third satellite reservoir includes oxidative methanogenic bacteria. Specific bacteria, either the acetoclastic bacteria or the oxidative bacteria, or predetermined quantities of both bacteria from each tank, are delivered to the AD tank depending on determinations made by the monitoring process or other process.

The AD satellite reservoir has a volume ratio to the AD tank within a range of 1:10 to 10:1. Alternatively, the volume of the AD satellite reservoir is approximately 1/100 or within a range of 1/200 to 1/2 of the volume of the AD tank.

Although reference is made specifically to the AD tank, the AD satellite reservoir and its principles may be applicable to other tanks and processes described herein and are not intended to be limited to the AD tank. For example, a satellite reservoir may be used to one or more of ASB tank, the AD tank, the mixing tank, and processes that occur during any phase related to reactions described herein.

Applying these findings, a synthetic microbial community may be incorporated in one or more of the ASB tank or in the AD tank. A synthetic microbial community is a systems approach to reduce the complexity, while increasing the controllability, by selecting genetically-engineered or wild type microorganisms that cooperate metabolically. The combination of different microbial species may decrease the potential competition between species often leading to the co-metabolism of a substrate in an independent manner. In the ASB tank, depending on feedstock, another species, Clostridium thermocellum may help decompose lignocellulolytic materials. The synthetic community in the AD tank is configured to digest acetate and lactate and/or the products of pretreatment that are substrate dependent.

An example kinetic model for ASB pretreatment results in ASB effluent, with acetate as the major pretreatment product.

The model includes the following reactions:

Cellulose(s)+H₂O(l)→glucose(aq)

and glucose(aq)+3OH⁻(aq)→3CH₃COO⁻(aq)+3H₂O(l)

d[CH₃COO⁻]/dt=−ΔGc=−(ΔG°+RTIn([CH₃COO⁻]³/[OH—]³[glucose])[CB]

d[glucose]/dt=k₄[active enzyme][substrate sites]−(1/3)d[CH₃COO⁻]/dt

d[CB]/dt=k₅[CB]→d[CB]/[CB]=k₅t→In([CB]/[CB]₀)=k₅(t−t₀)→[CB]ₜ=[CB]₀e^{k5(t-t0)}

d[substrate sites]/dt=a[(TSS)₀−(d(TSS)ₜ/dt)]

d[active enzyme]/dt=d[CB]/dt=(n_{CB0}e^{k7t})/V_{ASB}

An example kinetic model for a pretreated substrate may be used for the ASB effluent that enters the AD tank.

The model includes the following reactions:

CH₃COO⁻(aq)→CH₄(g)+HCO₃⁻(aq)

and VS→CH₄(g)+CO₂(g)

d[CH₃COO⁻Vₗ/dt=−d(P_{CH4}V_{g}/RT)/dt=Δ₈Gc=(Δ₈G°+RTIn(P_{CH4}[HCO₃⁻]/CH₃COO⁻]))[acetoclastic methanogens]

d[CH₃COO⁻]/dt=−k₈[CH₃COO⁻][acetoclastic methanogens]

d[VS]=−k₉[VS][oxidative methanogens]=d(2bP_{CO2}V_{g}/VₗRT)/dt

### SYMBOLS

S, l, aq=solid, liquid, gas phase
[ ]=concentration of substance
ΔnG=Gibbs energy change for reaction n
ΔnG°=standard Gibbs energy change for reaction n
c=conductance
R=gas constant
T=absolute (Kelvin) temperature
CB=C. bescii
kₙ=rate constant for reaction n
t=time
n_{CB0}=number of C. bescii cells at time=0
V_{ASB}=volume of ASB tank
Vₗ=liquid volume in AD tank
V_{g}=volume of biogas produced to time t
Pₓ=pressure of x
VS=volatile solids input to AD
TSS=total suspended solids input to ASB
a=cellulose sites per TSS
b=accessible sites per VS

Some possible results are anticipated as follows—
1. The C. bescii inoculum may not survive or grow as it should. In that case the treated and control AD tank results should be very close to the same.
2. If glucose is produced from cellulose much faster than C. bescii can digest it, then ASB effluent in the AD tank will produce gas at a higher rate than the control but the methane content will not be as high as it should be.
3. If the ASB tank is fed at too high a concentration of substrate, the C. bescii will grow to a stationary phase and stop making enzymes before pretreatment is complete. In this case, the biogas from the treated material will be greater and be produced faster and have a higher methane content, but will not get good carbon conversion to biogas.

This model suggests that pretreatment tanks, such as the ASB tank, should be filled according to

Vₜ=V₀e^{kt}

Vₜ=volume at time t

V₀=volume at time zero with C. bescii seed

t=time in hours

k=constant depending on doubling time of C. bescii on a particular substrate

| doubling | |
|---|---|
| time/hours | k/hours⁻¹ |
| 2 | 0.346574 |
| 2.5 | 0.277259 |
| 3 | 0.231049 |
| 3.5 | 0.198042 |
| 4 | 0.173287 |
| 4.5 | 0.154033 |
| 5 | 0.138269 |

Implementing this model should maintain an exponentially growing culture and maximize output of exozymes.

To further design the treatment, the following formulas may be helpful—

Gas composition from stoichiometry of carbon going into AD tank.

Cᵢₙ=2xCH₃COO⁻+3yCH₃CH(OH)COO⁻+6zC₆H₁₂O₆ (products of pretreatment)

Cₒᵤₜ=aCH₄+bCO₂+cHCO₃⁻ (products of methanogenesis)

x, y, z, a, b, and c are in units of moles of compound.
From the reactions of methanogenesis

xCH₃COO⁻+xH₂O=xCH₄+xHCO₃⁻yCH₃CH(OH)COO⁻+yH₂O=yHCO₃⁻+1.5yCH₄+0.5yCO₂

zC₆H₁₂O6 =3zCH₄+3zCO₂

a=x+1.5y+3z

b=0.5y+z

c=x+y

If x, y and z (acetate, lactate, and glucose) are measured and total C in the filtered solution coming from pretreatment, gas composition and bicarbonate production can be predicted.

### Recycling

During pretreatment in the ASB tank, the pH level naturally drops and becomes acidic. Bicarbonate ions that are formed in the AD tank may be removed from the AD tank and put back into the ASB tank, which neutralizes the pH level of the ASB tank. In this manner, matter in the AD tank is recycled in the ASB tank. This act makes it unnecessary to buy a base to neutralize the pH level, and is thus a cost-saving step. In another variation, an ammonia scrubber is included in the system to scrub contents that are recycled from the AD tank. The scrubber strips out ammonia and keeps the concentration below a toxic level. A heat exchanger or solar energy may also be used to heat the contents being recycled from the AD tank to the ASB tank.

### FIGURES

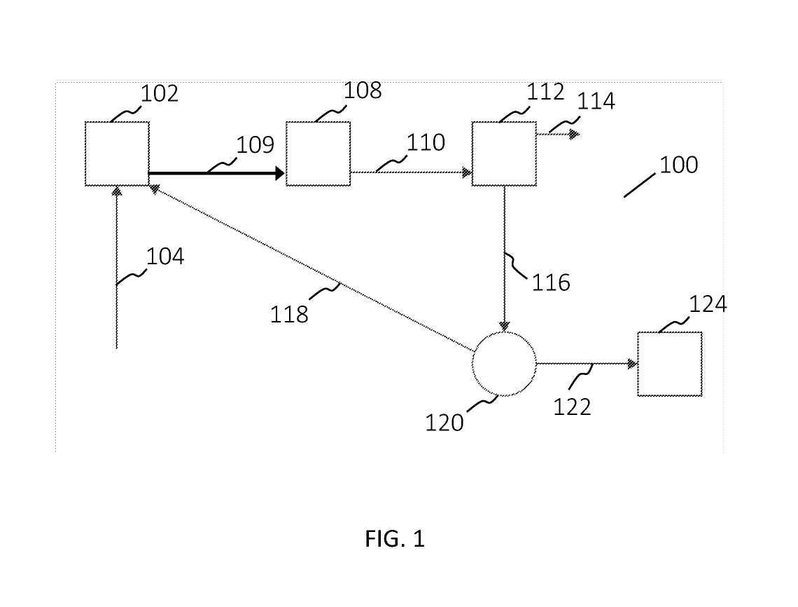

Examples are provided followed by reference to FIGS. 1-9 in relation to principles discussed herein.

An example reactor for conversion of biomass into biogas comprises a pretreatment ASB tank containing anaerobic organisms and an AD tank that receives ASB tank effluent. The ASB tank receives biomass effluent that includes unsolubilized lignocellulosic components and treats the biomasss effluent under conditions such that the anaerobic organisms reproduce and solubilize the lignocellosic components. Upon receiving the ASB tank effluent, the AD tank contains anaerobic bacteria that convert organism metabolic products of the lignocellulosic components into biogas under anaerobic digestion conditions. Outputs from the AD tank including the biogas and a slurry of undigested biomass.

An example reactor may further comprise a mixing tank where the biomass is mixed with water, heated, and components in the biomass are solubilized. The effluent of biomass suspended in water from the mixing tank is then transferred to the ASB tank.

An example reactor may further comprise one or more satellite reservoirs that provide one or more of a bacteria, nutrient, and other content discussed herein, to one or more of the ASB tank and AD tank according to principles discussed herein.

An example reactor may further comprise an AD tank that contains anaerobic bacteria and that receives contents comprising solubilized biomass. The anaerobic bacteria is to convert organism metabolic products of lignocellulosic components of the solubilized biomass into biogas under anaerobic digestion conditions. Outputs from the AD tank include the biogas and a slurry of undigested biomass. An AD satellite reservoir is used to supply one or more microbial species to the AD tank to support conversion of the organism metabolic products.

An example method for converting biomass into biogas comprises treating biomass in an ASB environment with anaerobic organisms that solubilize and metabolize lignocellulosic components of the biomass. The treated biomass from the ASB environment is further treated within an AD environment with anaerobic bacteria that convert products of the lignocellulosic components from the ASB environment into biogas under anaerobic digestive conditions.

An example method may further comprise providing conditions to produce bicarbonate in the AD environment. The bicarbonate may be recycled to the ASB environment.

An example method may further comprise mixing and heating the biomass with water before treating the biomass in the ASB environment. An example method may further comprise recovering heat from one of more of the AD environment and the ASB environment.

An example method may further comprise buffering the ASB environment to produce acetate and lactate as part of the solubilized components.

An example method may further comprise favoring ASB acetate ion production over lacate ion production within the ASB environment to produce a reduced CO₂ stream in the AD environment.

An example method of converting biomass into biogas comprises treatment of biomass in an ASB environment with organisms that solubilize and metabolize lignocellulosic components of the biomass with minor solubilization by chemical treatment or by mechanical treatment with predominant solubilization by the organisms. The method further includes treatment of the treated biomass from the ASB environment in an AD environment with anaerobic bacteria that convert products of the lignocellulosic components from the ASB environment into biogas under anaerobic digestive conditions.

Large scale production of converting biomass into biogas is anticipated based on examples and principles discussed herein.

Turning to FIG. 1, a reactor 100 is shown including a mixing tank 102, ASB tank 108, and AD tank 112. Biomass 104 is supplied to the mixing tank 102. Mixing tank effluent produced by the mixing tank 102 is delivered to the ASB tank 108 for pretreatment. ASB effluent produced within the ASB tank 108 is forwarded to an AD tank 112 for anaerobic digestion. Reactions therein produce biogas CH₄ and CO₂ 116 to be used in making gas for a pipeline, or electrical generation 124. The CH₄ and CO₂ 116 may go through gas processing 120 prior to being used in making gas for a pipeline or electrical generation 124. Additionally, the CH₄ and CO₂ may go through gas processing and then the CO₂ be recycled to the mixing tank 102 for treatment of biomass.

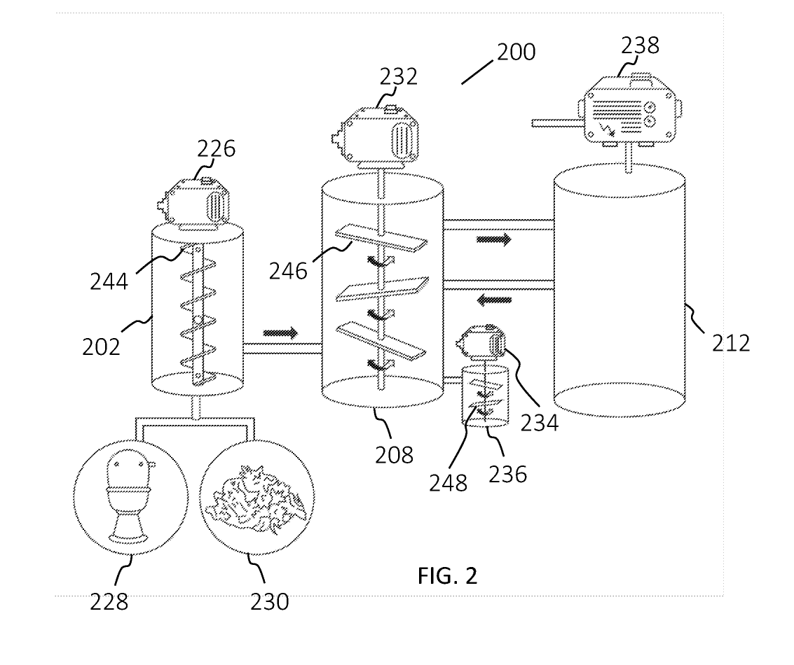

Turning to FIG. 2, an example reactor is shown in which biosolids 228 and green waste 230 are supplied to a mixing tank 202. The contents within the mixing tank 202 are mixed with a mixer represented by paddles 244. Also, a mixing motor 226 is shown as powering the mixing tank 202 process. Mixing tank effluent is produced and supplied to the ASB tank 208 as indicated by a black arrow. The ASB tank 208 includes a mixer represented by paddles 246. A mixing motor 232 is shown as mixing the ASB tank 208. ASB tank effluent that is produced is supplied to the AD tank 212 for anaerobic digestion as indicated by a black arrow. CO₂ and bicarbonate that are produced in the AD tank 212 may be recycled back to the ASB tank as indicated by a black arrow.

Alternatively, the biogas may be transferred to an outside source. In an example operation, at least a portion of the biogas methane is burned by an AD electrical generator 238. In this manner, the AD tank may provide power to the system. In another example, at least a portion of the biogas methane is separated from the AD tank and used as one or more precursors for synthetic processes. Examples of synthetic processes include oxygenated aromatic compounds for synthesis of medicines and new materials.

Separation of the treated biomass or at least a portion of the treated biomass from the ASB environment can occur by one or more of semi-permeable membranes, centrifuge purification, distillation, filtration, industrial chromatography with zeolites, and sorption. Other known mechanical and chemical means are anticipated. Although reference is made to FIG. 2, the use of biogas methane and separation as discussed herein applies to all examples described throughout the specification.

Although not shown, the AD tank 212 may include a mixer, including a mixer like the mixing tank 202 or ASB tank 208. Variations of power are anticipated for the various tanks and reactors herein.

An ASB satellite reservoir 236 is shown being functionally connected to the ASB tank 208 and powered by an ASB motor 234. Satellite paddles 248 as shown may be used to stir one or more of bacteria, nutrients, and other matter to facilitate the pretreatment process within the ASB tank 208.

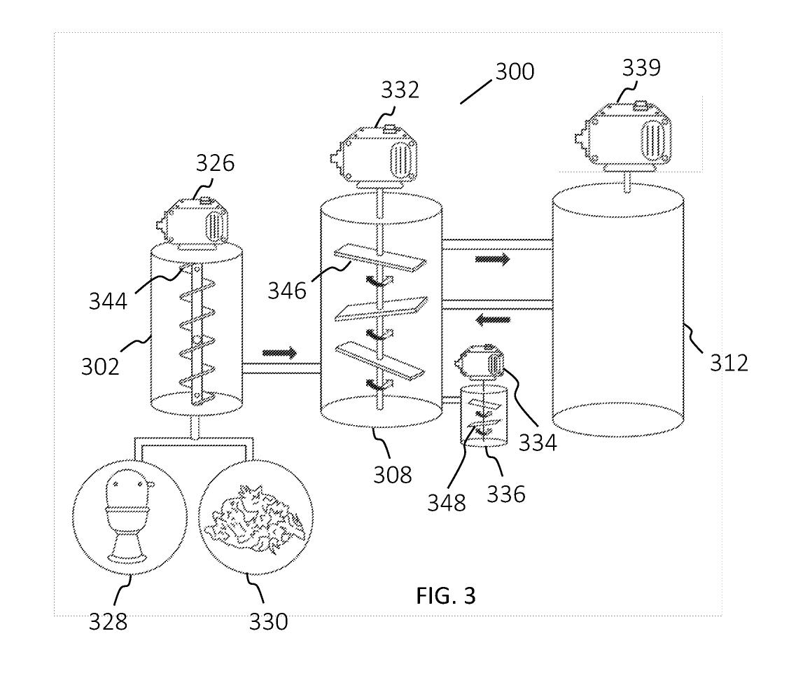

Alternatives include no power generation with an electrical generator 238. With or without an electrical generator, a motor may be used with one or more of the various tanks discussed herein. Such an example that replaces the electrical generator 238 with a motor 339 is shown in FIG. 3. The other components are shown as the same. Namely, the example reservoir is shown in which biosolids 328 and green waste 330 are supplied to a mixing tank 302. The contents within the mixing tank 302 are mixed with a mixer represented by paddles 344. Also, a mixing motor 326 is shown as powering the mixing tank 302 process. Mixing tank effluent is produced and supplied to the ASB tank 308 as indicated by a black arrow. The ASB tank 308 includes a mixer represented by paddles 346. A mixing motor 332 is shown as powering the ASB tank 308. ASB tank effluent that is produced is supplied to the AD tank 312 for anaerobic digestion as indicated by a black arrow. An ASB satellite reservoir 336 is shown being functionally connected to the ASB tank 308 and powered by an ASB motor 334. Satellite paddles 348 as shown may be used to stir one or more of bacteria, nutrients, and other matter to facilitate the pretreatment process within the ASB tank 308.

CO₂ and bicarbonate that are produced in the AD tank 312 may be recycled back to the ASB tank as indicated by a black arrow. The AD tank is powered by motor 339.

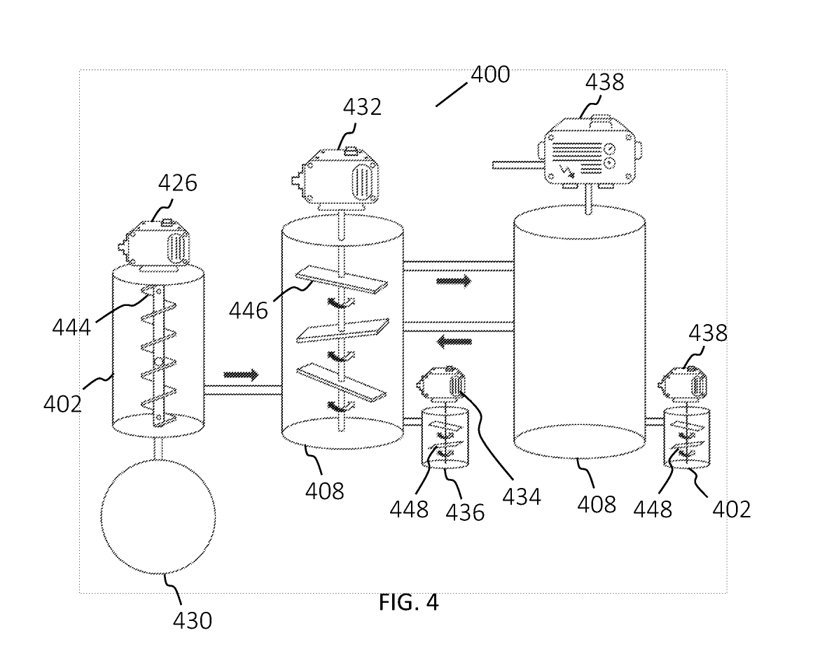

Turning to FIG. 4, biomass 430 including biosolids, manure, green waste, food waste, etc. is supplied to mixing tank 402 for mixing by mixing paddles 444 as powered by mixing motor 426. Mixing tank effluent that is produced is supplied as indicated by a black arrow to ASB tank 408 for pretreatment that includes mixing paddles 446 as powered by ASB motor 432. ASB effluent that is produced is supplied to AD tank 408 as indicated by a black arrow for anaerobic digestion. CO₂ and bicarbonate that are produced may be recycled back to the ASB tank as indicated by a black arrow or used for other uses as discussed previously. An ASB satellite reservoir 436 is used to supply one or more of bacteria, nutrient, or other matter to the ASB tank 408 as mixed by mixing paddles 448 and powered by ASB satellite motor 434. A second reservoir, an AD satellite reservoir 448 is used to supply acetoclastic consortium or other matter to the AD tank 408 as mixed by mixing paddles 448 and powered by AD satellite motor 438.

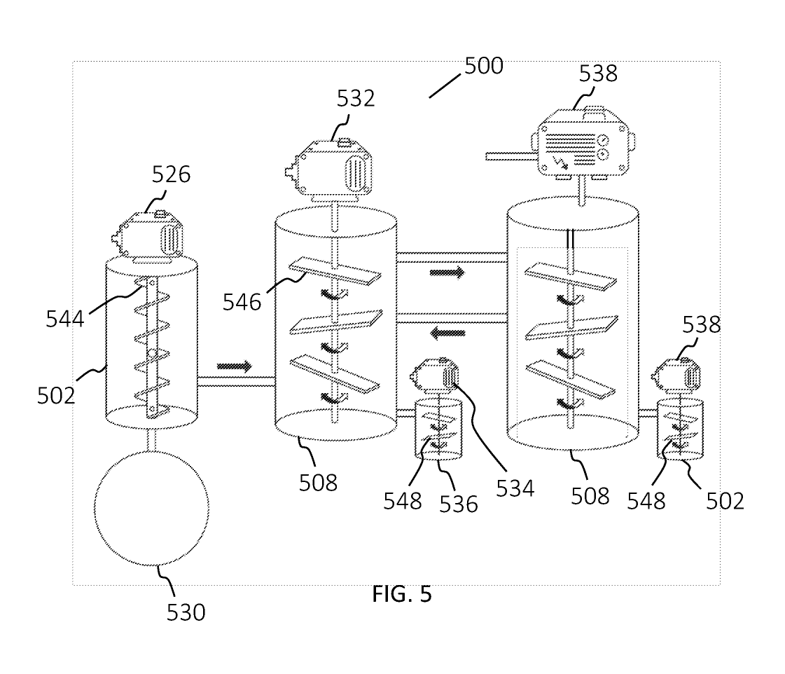

The AD tank may include mixing capabilities such as mixing paddles used for the mixing tank or the ASB tank. FIG. 5 shows the same reactor but the AD tank includes mixing paddles.

Biomass 530 including biosolids, manure, green waste, food waste, etc. is supplied to mixing tank 502 for mixing by mixing paddles 544 as powered by mixing motor 526. Mixing tank effluent that is produced is supplied as indicated by a black arrow to ASB tank 508 for pretreatment that includes mixing paddles 546 as powered by ASB motor 532. ASB effluent that is produced is supplied to AD tank 508 as indicated by a black arrow for anaerobic digestion. CO₂ and bicarbonate that are produced may be recycled back to the ASB tank as indicated by a black arrow or used for other uses as discussed previously. An ASB satellite reservoir 536 is used to supply one or more of bacteria, nutrient, or other matter to the ASB tank 508 as mixed by mixing paddles 548 and powered by ASB satellite motor 534. A second reservoir, an AD satellite reservoir 548 is used to supply acetoclastic consortium or other matter to the AD tank 508 as mixed by mixing paddles 548 and powered by AD satellite motor 538.

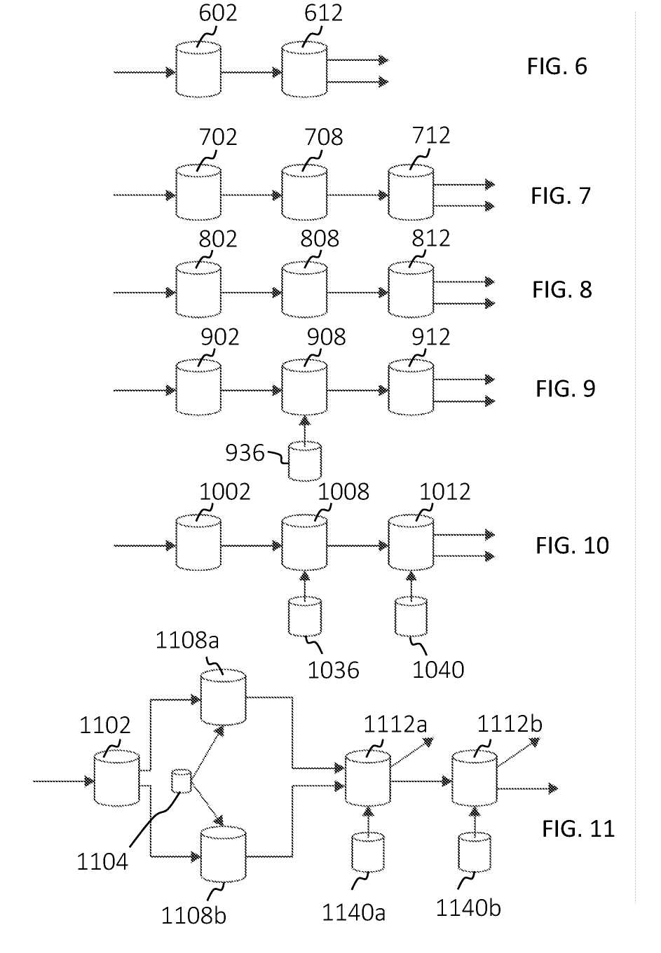

Turning to FIGS. 6-11, variations of a reactor are shown that may be used to incorporate principles discussed herein.

In FIG. 6, a reactor is shown that includes a mixing tank 602 and an AD tank 612.

Turning to FIG. 7, a reactor is shown that includes a mixing tank 702 that only mixes without heat, an ASB tank 708, and an AD tank 712.

Turning to FIG. 8, a reactor is shown that includes a mixing tank 802 that mixes and heats, an ASB tank 808, and an AD tank 812.

Turning to FIG. 9, a reactor is shown that includes a mixing tank 902, an ASB tank 908 that includes an ASB satellite reservoir 936, and an AD tank 912.

Turning to FIG. 10, a reactor is shown that includes a mixing tank 1002, an ASB tank 1008 that includes an ASB satellite reservoir 1036, and an AD tank 1012 with an AD satellite reservoir 1040.

Turning to FIG. 11, a reactor is shown that includes a mixing tank 1102, two ASB tanks 1108a and 1108b, an ASB satellite reservoir 1104 that provides one or more of bacteria, trace nutrients, or other matter to the two ASB tanks 1108a and 1108b, and two AD tanks 1112a and 1112b, each AD tank having its own respective AD satellite reservoir 1140a and 1140b.

Variations further include that no mixing tank be used. For example, the examples illustrated in FIGS. 6-11 may be used without a mixing tank. Other reactors are anticipated that incorporate principles discussed herein.

### Dairy Manure Testing Results

A 6% solids solution of dairy manure was pretreated in the ASB tank for 48 hours. After pretreatment, the material was anaerobically digested and the rate of biogas production and composition was measured. A control experiment was conducted by heating a 6% solids solution of dairy manure to 75° C. for 48 hours. Afterward, the solution was anaerobically digested and the rate of biogas production and composition was measured. FIG. A reproduced below shows pretreated manure (solid black line) produced 2.5× more biogas than the control (dashed black line). FIG. B reproduced below shows the rate of biogas production. The maximum rate of biogas production from pretreated manure (filled black circles) was 2.7× larger than the maximum rate of biogas production from the control (filled black triangles). The methane content in pretreated, anaerobically digested manure was 74% compared to 72% for the control.

### Waste Activated Sludge (WAS) Testing Results

A 5% solids solution of WAS was pretreated in the ASB for 48 hours. After pretreatment, the material was anaerobically digested and the rate of biogas production and composition was measured. A control experiment was conducted by heating a 6% solids solution of WAS to 75° C. for 48 hours. Afterward, the solution was anaerobically digested and the rate of biogas production and composition was measured. FIG. A reproduced below shows pretreated WAS (solid black line) produced 2.4× more biogas than the control (dashed black line). FIG. B reproduced below shows the rate of biogas production. The maximum rate of biogas production from pretreated WAS (filled black circles) was 2.6× larger than the maximum rate of biogas production from the control (filled black triangles). The methane content in pretreated, anaerobically digested WAS was 76% compared to 62% for the control.

While this invention has been described with reference to certain specific embodiments and examples, it will be recognized by those skilled in the art that many variations are possible without departing from the scope and spirit of this invention, and that the invention, as described by the claims, is intended to cover all changes and modifications of the invention which do not depart from the spirit of the invention.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method of converting biomass into biogas, comprising:
treating biomass in an ASB environment with anaerobic organisms that solubilize and metabolize components of the biomass; and
treating the treated biomass from the ASB environment in an AD environment with anaerobic bacteria and archea that convert products from the ASB environment into biogas under anaerobic digestive conditions.

34. The method of claim 33, wherein the ASB environment is separate from the AD environment as part of a two compartment system.

35. The method of claim 33, further comprising:
mixing and heating the biomass with water before treating the biomass in the ASB environment to mitigate pH changes and reduce oxygen concentration in an the ASB environment.

36. The method of claim 33, further comprising:
recycling bicarbonate or other base produced in the AD environment to the ASB environment.

37. The method of claim 33, further comprising:
separating at least a portion of the ASB treated biomass to be purified and used for another process.

38. The method of claim 33, further comprising:
separating at least a portion of the ASB treated biomass to be used as one or more precursors for synthetic processes rather than anaerobic digestion.

39. The method of claim 33, further comprising:
separating at least a portion of the treated biomass from the ASB environment by one or more of semi-permeable membranes, centrifuge purification, distillation, filtration, industrial chromatography with zeolites, and sorption.

40. The method of claim 33, further comprising buffering a pH of the ASB environment to produce acetate and lactate as part of solubilized components that are produced.

41. The method of claim 33, further comprising:
buffering a pH of the ASB environment to maintain an optimal culture with one or more genera of Caldicellulosiruptor, Clostridium, Thermoanaerobacterium saccharolyticum, or other hyperthermophilic anaerobic bacteria.

42. The method of claim 33, further comprising:
favoring production of acetate ion over lactate ion production within the ASB environment to produce a reduced CO₂ content in the biogas from the AD environment.

43. The method of claim 33, further comprising:
containing the biomass and the organisms in the ASB environment for a period of time between 12 to 48 hours, 2 days to 4 days, or 4 days to 7 days.

44. The method of claim 33, further comprising:
maintaining the ASB environment with organisms so as to facilitate production of enzymes for solubilizing biomass.

45. The method of claim 33, further comprising:
providing an ASB environment that has a volume ratio of 1:10 to 10:1 with the AD environment as defined by a relative rate of degradation of the biomass in the ASB tank to the production of biogas in the AD tank.

46. The method of claim 33, further comprising:
treating the ASB environment separate from the AD environment as part of a two compartment system, and
treating the system as one or more of a batch process, semi-continuous process, and a continuous process.

47. The method of claim 33, further comprising:
pretreating the biomass before it enters the ASB environment.

48. The method of claim 33, further comprising:
maintaining in an external reactor one or more hyperthermophillic anaerobic bacteria for the ASB environment, with the substrate comprising one or more of nutrients for growth, food waste, animal manure, biosolids, waste organic material, sewage, garbage, waste activated sludge, FOG, waste paper, lignocellulosic plant materials, and cellular material.

49. The method of claim 33, further comprising:
maintaining in an external reactor one or more acetoclastic methanogens for the AD environment, with the substrate comprising the effluent from the ASB or ASB effluent and nutrients for growth, food waste, animal manure, biosolids, waste organic material, sewage, garbage, waste activated sludge, FOG, waste paper, lignocellulosic plant materials, and cellular material.

50. A method of converting biomass into biogas, comprising:
treating biomass in an ASB environment with organisms that solubilize and metabolize components of the biomass prior to and with minor solubilization by chemical treatment or by mechanical treatment with predominant solubilization by the organisms; and
treating the treated biomass from the ASB environment in an AD environment with anaerobic bacteria that convert products of the lignocellulosic components from the ASB environment into biogas under anaerobic digestive conditions.
